(19) United States
(12) Patent Application Publication
(10) Pub. No.: US 2005/0053677 A1
(43) Pub. Date: Mar. 10, 2005

(54) Botanical extractions process

(76) Inventor: John Bertram Greene, Halswell Christchurch (NZ)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/493,961

(22) PCT Filed: Oct. 31, 2002
(86) PCT No.: PCT/NZ02/00230

(30) Foreign Application Priority Data
Oct. 31, 2001 (NZ) 515182

(51) Int. Cl. A61K 35/78

(52) U.S. Cl. 424/725; 424/728; 424/729; 424/730; 424/778; 424/764; 424/766; 424/770

(57) ABSTRACT

The present invention discloses a method of extracting biologically active compounds from botanic material, the method stabilising the botanical material from oxidative degradation. This preserves the biologically active compounds in the material. In the main embodiment, the plant material is mixed with a solution containing at least one acid and one antioxidant. It has been found that the oxidative degradation of the biologically active compounds in plant material can be prevented, slowed or stopped by the invention.

### TECHNICAL FIELD

The present invention relates to an improved botanical extraction process. The invention more specifically relates to an improved botanical extraction process that prevents degradation of biologically active compounds.

### BACKGROUND ART

A number of biologically active compounds occur in flora, particularly the leafy portions, in generally small quantities. For example, biologically active compounds (such as polyphenolics) in the leaf, roots, fruit and flowers can be extracted from Echinacea. Other compounds (alkamides, fructofuranosides and arabinogalactans) are also capable of extraction from Echinacea. Further, additional biologically active compounds (for example bioflavonoids, polysaccharides, anti-oxidants, alkaloids, saponins, isoflavones, (etc)) are extracted commercially from many plant materials.

One key difficulty with commercial extraction of biologically active compounds is that the active compound(s) are unstable and, in particular, are prone to oxidation which degrades the compounds in the extract.

One example of an extraction process is the solvent extraction of polyphenolics, fructofuranosides and alkamides from Echinacea purpurea. Most commercial extracts are prepared with an ethanol-water mixture which usually contains 25-70% ethanol.

Whilst solvent extraction techniques are more consistent in quality than water extraction techniques, they have the disadvantage that sometimes the solvents are undesired. Such solvents add to the processing cost, are sometimes difficult to handle, and can remove the ‘natural’ image of the product. Furthermore the use of solvents does not address the problem of oxidative degradation of the active compounds in the extract. Water extraction techniques, whilst more ‘natural’ than use of solvents, can be less successful at extraction.

Degradation of the biologically active compounds occurs via a number of possible oxidation reactions. It is generally thought that polyphenolics and the polysaccharides found in, for example, Echinacea plant material provide part of the medicinal activity. As these polyphenolics are released in the extraction process, enzymes and polyphenolic oxidase (PPO) in particular, cause an oxidation reaction to occur. The PPO enzyme appears to be highly active in Echinacea with research showing an almost 80% reduction in phenolic levels within 5 minutes. Other oxidation reactions also occur but the above is the fastest to degrade the biological compounds of the extract

This degradation of biologically active compounds during extraction is sometimes further exacerbated by degradation during drying and/or storage of plant material prior to the extraction process, when fresh material is not used in the extraction process.

U.S. Pat. No. 6,217,878 attempts to address the problem of oxidative degradation by utilising heat at the start of the process to denature the PPO enzymes causing the oxidation reaction. This is a blanching type operation. While heat destroys the enzyme which catalyses the oxidation reaction, further problems are caused including heat degradation of the phenolic compounds and other active compounds. As a result, final phenolic activity is reduced. The invention also requires the use of a carrier compound (tricalcium phosphate) in reasonably large quantities thus introducing new compounds to the method.

The above discussion has been directed to that of Echinacea extraction. It will be appreciated by those skilled in the art that the principles of degradation of biologically active compounds in all plant material are well illustrated by this plant.

It is an object of the present invention to address the above problems by providing a method of extraction of compounds from plant material that avoids oxidative degradation of biologically active compounds in the plant material.

It is a further object of the present invention to provide a method of extraction that is cost effective.

It is still a further object of the invention to disclose a method that addresses pH control in extract processing.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

Ascorbic acid is a white, crystalline vitamin, C₆H₈O₆, found in citrus fruits, tomatoes, potatoes, and leafy green vegetables and used to prevent scurvy. It is also called vitamin C. It is a known as an antioxidant in the food industry.

Citric acid is an organic acid containing three carboxyl groups and has the chemical formula of C₆H₈O₇. It is found in citrus fruits (oranges, grapefruit, lemons). It is used in commercially processed foods and can complex some metals that act as catalysts in oxidation.

### DISCLOSURE OF INVENTION

According to one aspect of the present invention there is provided a method of extracting biologically active compounds from botanical material, said method including the step of:
(a) mixing together the botanical material and a solution including at least one acid and at least one antioxidant to form a mixture;
   characterised in that the combination of an acid and an antioxidant substantially prevents, slows and/or halts oxidative degradation of compounds in the mixture.

According to a further aspect of the present invention there is provided a method of extracting biologically active compounds from botanical material substantially as described above, said method further characterised in that the use of the solution in step (a) prevents, slows and/or halts polyphenolic oxidation reactions in the mixture.

According to a further aspect of the present invention there is provided a method of extracting biologically active compounds from botanical material substantially as described above, said method further characterised in that the use of the solution in step (a) reverses the polyphenolic oxidation reaction equilibrium.

According to another aspect of the present invention there is provided a method of extracting biologically active compounds from botanical material substantially as disclosed above, wherein the solution of step (a) is added to the botanical material by means selected from the group: spraying; dipping; pouring over, and a combination thereof.

According to a further aspect of the present invention there is provided a method of extracting biologically active compounds from botanical material substantially as described above, wherein the solution in step (a) is food grade quality. Preferably, the solution is a mixture selected from the group including: citric acid; ascorbic acid; cysteine; cinnamic acid; sulphur dioxide; vitamin E; and any combination thereof.

According to a further aspect of the present invention there is provided a method of extracting biologically active compounds from botanical material substantially as described above, wherein the solution in step (a) includes hexyl resorcinol

According to a further aspect of the present invention there is provided a method of extracting biologically active compounds from botanical material substantially as described above; wherein the solution of step (a) is: 0.5-30% (by weight) citric acid; 0.5-30% (by weight) ascorbic acid; and water. Optionally, the acid components are added as a powder or a prepared liquid.

According to a further aspect of the present invention there is provided a method of extracting biologically active compounds from botanical material substantially as described above, wherein the solution in step (a) includes water that has been deoxygenated and/or chilled to between 1 and 4° C.

According to a further aspect of the present invention there is provided a method of extracting biologically active compounds from botanical material substantially as described above, wherein the material is selected from the range of plants known to be used for herbal medicines and natural remedies. Preferably, said plant material is prone to oxidative reactions that reduce the activity of the material.

Most preferably, said botanical material is selected from the group including: Echinacea; Ginkgo Biloba; Kava Kava; Ginseng; Black Cohosh; Green Tea; St John's Wort (with hypercerin as an active); Artichoke; Chamomile; Dong Quai; Grape Seed; Grape Skins; berries; Hawthorn; Hops; Passion Flower; Pine Bark; Red Clover, Olive leaf; currants; and combinations thereof.

Preferably, the material selected is the leafy portion of a plant, free of stems and branches and/or twigs. It be appreciated, however, that any part of the plant, including flower heads, or roots may be used.

Optionally, the botanical material is the by product or pressate of other plant processing extraction steps. Optionally, the plant material is the by product of, for example, a seed extraction, a super critical fluid extraction (SCFE), or the by product of flower harvesting.

According to a further aspect of the present invention there is provided a method of extracting biologically active compounds from botanical material substantially as described above, wherein step (a) occurs immediately before harvest of the plant material.

According to a further aspect of the present invention there is provided a method of extracting biologically active compounds from botanical material substantially as described above, said method including a step before step (a) of:
(xa) disintegrating the botanical material.

Preferably, the step of disintegration occurs within a time of less than 5 minutes before step (a). Most preferably this time is less than 10 seconds.

According to a further aspect of the present invention there is provided a method of extracting biologically active compounds from botanical material substantially as described above, wherein step (xa) is simultaneous with step (a).

In a preferred embodiment, the plant material disintegrated is fresh material. Optionally, the material is frozen prior to disintegration.

Preferably, the botanical material is disintegrated to a size of 0.1 mm to 50 mm in length. Preferably, disintegration is completed by a method selected from the group including: rubbing; milling; chopping; and combinations thereof.

According to a further aspect of the present invention there is provided a method of extracting biologically active compounds from botanical material, said method further including the step, after step (a) of:
(b) separating the liquid from the residue;

Preferably, step (b) is completed at any time between immediately after step (a) and 7 days after step (a).

Optionally also, during the contact time period described above, the botanical mixture is periodically or continuously stirred, mixed or otherwise agitated.

According to another aspect of the present invention there is provided a method of extracting biologically active compounds from botanical material substantially as disclosed above, wherein step (b) is completed by: collecting a liquid extract as the retentate of reverse osmosis; collecting a liquid extract as the retentate of ultra-filtration, or collecting a liquid extract as the retentate of a combination of these methods.

According to another aspect of the present invention there is provided a method of extracting biologically active compounds from botanical material substantially as disclosed above, wherein in step (b) the method of separation to collect a liquid extract is selected from the group including: filtration; super critical fluid extraction (SCFE); mechanical de-watering; and any combination thereof.

Most preferably the method of separation is a combination of filtration and mechanical de-watering.

According to a further aspect of the present invention there is provided a method of extracting biologically active compounds from botanical material substantially as described above wherein said method includes a further step, after step (b) of:
(c) drying the liquid extract from step (b) to obtain a solid extract.

According to another aspect of the present invention there is provided a method of extracting biologically active compounds from botanical material substantially as described above, wherein said method includes a further step, after step (b) of:
(d) pressing the residual plant material to obtain a pressate which is then combined with the liquid extract of step (b).

According to another aspect of the present invention there is provided a method of extracting biologically active compounds from botanical material substantially as described above, wherein said method includes a further step, after step (d) of:
(e) filtrate and pressate drying.

The filtrate and or pressate (if present) are preferably dried by freeze drying, spray drying or other known drying techniques.

According to a yet further aspect of the present invention there is provided a method of extracting biologically active compounds from botanical material substantially as described above, wherein any one or a combination of steps described above are conducted in the absence of oxygen. Optionally, a nitrogen atmosphere is used.

According to a yet further aspect of the present invention there is provided a method of extracting biologically active compounds from botanical material, as claimed in any preceding claim, wherein the above described method includes the further step, after step (a) and the sequences of steps selected from steps (b), (c), (d), or (e), said step being:
(f) one or more further cycles of at least one of steps (a), (b), (c), (d), or (e).

According to a yet further aspect of the present invention there is provided a method of extracting biologically active compounds from botanical material substantially as described above, wherein the steps up to and including obtaining the liquid extract are conducted at the harvest site.

According to a further aspect of the present invention there is provided a method of extracting biologically active compounds from botanical material substantially as described above, wherein the phenolic level after extraction is maintained at 50-100% as compared with the starting levels, measured as dried extract. Preferably, the phenolic level after extraction is maintained at 70-100% of starting levels.

According to another aspect of the present invention there is provided a product formed by the process as described above wherein the product is the mixture from step (a).

According to another aspect of the present invention there is provided a product formed by the process as described above wherein the product is the liquid extract from step (b).

According to another aspect of the present invention there is provided a product formed by the process as described above wherein the product is the residue from step (b).

According to another aspect of the present invention there is provided a product formed by the process as described above wherein the product is the mixture from step (d).

According to another aspect of the present invention there is provided a product formed by the process substantially as described above, wherein the product of the process is a solid.

It has been found to be advantageous to use only the leafy part of botanical material with the above method for extraction of polyphenolics. However, it is to be noted that biologically active compounds can concentrate in any part of a plant. Thus the selection of a part of a plant to be extracted advantageously over others, will be dependent upon the compound to be extracted(s) and the plant from which it is extracted.

It will be known to those skilled in the art that biologically active compounds can deteriorate if biological material is dried or is picked in poor condition etc. The use of the above described method, by taking botanical material direct from harvest as described, ensures that there is very little opportunity for degradation of the biologically active compounds.

The advantage of the invention as described above is that the oxidative degradation of the biologically active compounds in plant material can be prevented or stopped or slowed as soon as the degradation starts to occur.

Aspects of the present invention will be apparent from the following examples which are given by example only.

### BEST MODES FOR CARRYING OUT THE INVENTION

### EXAMPLE 1

Take 400 kg of frozen E. purpurea tops and pass through a granulator and re-fireeze. Note that freezing is not essential.

Prepare solution by mixing 160 g of citric acid with 220 g of ascorbic acid, dilute with water to 1000 ml and mix thoroughly.

### EXAMPLE 1.1

Take 10 kg of frozen E. purpurea and spray with 1 L of solution. Mix together and thaw to 15° C. or more. Add 19 litres of water to the mixture and stand with occasional stirring for approximately 2 hours.

Using a filter bag, filter the mixture and separate out the solid and liquid extract. Optionally, freeze the liquid extract for storage at this point or continue into the next step of evaporation to concentrate up to 50% total solids. Freeze down the remaining extract and freeze dry to collect a final powdered Echinacea extract.

### EXAMPLE 1.2

Take 5 kg of frozen granulated E. purpurea tops and mince. Add 500 ml of solution prepared as above.

Add water, press and filter as in Example 1.1 above. Freeze the collected extract and freeze dry.

### EXAMPLE 1.3

Take 1 kg of frozen granulated E. purpurea tops and blend. Add 100 ml of solution and mix thoroughly.

Add water, press and filter as in Example 1.1 above. Freeze the collected extract and freeze dry.

### EXAMPLE 2

25 tonnes of raw E. purpurea material is added into a mill. The mill of known construction, mills or chops the raw Echinacea bringing particles down to 10-20 mm, followed by a rubbing action mill similar to that used for grain.

As soon as particles emerge from the mill they are sprayed with a citric and ascorbic acid solution. The solution is a mixture of 400 kg of citric acid, 550 kg of ascorbic acid and then diluted using water. A small amount of thickener may optionally be added to the solution to ensure that the solution ‘sticks’ to the Echinacea material.

After this, the Echinaceal acid mixture can be further processed safely via known techniques including as pressing, filtration, pasteurisation, chilling, freezing, evaporation and drying.

In the above process a 2.5% starting phenolic level (dry basis by weight by HPLC analysis) is concentrated up to a 75% phenolic recovery. A 6.8% fructofuranoside level (dry basis by weight) is concentrated up to an 80% recovery as well

### EXAMPLE 3

Comparison of phenolic activity from standard water extraction methods without acid treatment compared to acid treatment methods.

The water extraction method was used as a control measure. Water extraction was used as per the methods above but without the addition of any citric or ascorbic acids.

Citric and ascorbic acids were added to the dilution water used to submerge the plant material before disintegration. The concentration of citric acid in the dilution water was 0.64 wt %, while that for ascorbic acid was 0.18 or 0.88 wt %, respectively. The plant slurry contained 6.3% dry plant material. The final pH of the extract ranged from 3.6 to 4.

The results were as follows:

| | Extract | | | | Yield of phenolics | | |
|---|---|---|---|---|---|---|---|
| | yield | Phenolics in extract | | | (% of phenolics in plant | | |
| | (% of dry | (% w/w of extract) | | | compared with base levels) | | |
| Sample | plant) | Caftaric | Cichoric | Total | Caftaric | Cichoric | Total |
| Water (control) | 35 | 0.03 | 0.06 | 0.09 | 2 | 1 | 1 |
| Citric acid (33.3 mM) | 46 | 0.9 | 1.0 | 1.9 | 83 | 28 | 41 |
| Citric (33.3 mM) + | 60 | 1.6 | 3.4 | 4.9 | 179 | 122 | 134 |
| Ascorbic acid | | | | | | | |
| (50 mM) | | | | | | | |

Notes:
Results based on HPLC analysis
Freeze-dried extracts were analysed
Freeze-dried plant contained 0.5% caftaric acid and 1.7% chichoric acid, thus a total of 2.2% phenolics

As is demonstrated above the phenolic levels in the extract are higher for citric acid alone and significantly higher for the citric and ascorbic acid combination. Similarly the percentage yield of phenolics in the plant are significantly higher, particularly for the citric and ascorbic acid combination where levels above 100% recovery are achieved. This suggests that the oxidation reaction is being reversed and oxidative phenolics in the extract initially are being reversed back to un-oxidised phenolics. It is appreciated that the combination of citric and ascorbic acids creates a synergistic mixture that greatly enhances the phenolic yield in botanical extracts. The mixture also co-extracts fructofuranosides effectively.

Aspects of the present invention have been described by way of example only and it should be appreciated that modifications and additions may be made thereto without departing from the scope thereof as defined in the appended claims.

1. A method of extraction of at least one biologically active phenolic compounds from a botanical material including the steps of:
(a) reducing the particle size of a botanical material;
(b) mixing the reduced particle size botanical material of step (a) with a solution containing water; an acid compound, and an anti-oxidant compound, wherein the acid and anti-oxidant compounds are different compounds, to produce a liquid extract and a solid residue; and
(c) separating the resulting liquid extract from the solid residue;
wherein said steps are carried out at a temperature not in excess of about 38° C.;
wherein the solution of step (b) has the property of substantially slowing, or halting oxidative degradation of said phenolic compounds and of retaining biological activity in the liquid extract,
wherein the liquid extract is adapted to be used in medicaments.

2. A method as claimed in claim 1, wherein the botanical material used in step (a) is freshly harvested.

3. A method as claimed in claim 1, wherein the botanical material used in step (a) is frozen harvested material.

4. A method as claimed in, claim 1 wherein said botanical material is at least one selected from the group consisting of: Echinacea; Ginkgo Billow; Kava Kava; Ginseng; Black Cohosh; Green Tea; St John's Wort (with hypercerin as an active); Artichoke; Chamomile; Dong Quai; Grape Seed; Grape Skins; Hawthorn; Hops; Passion Flower; Pine Bark; Red Clover; Olive Leaf; currants; berries; and a combination thereof.

5. A method as claimed in claim 1, wherein the botanical material selected is the leafy portion of a plant, free of stems and branches and/or twigs.

6. A method as claimed in claim 1, wherein the botanical material particle size is reduced to a size of about 0.1 mm to 50 mm in length.

7. A method as claimed in claim 1, wherein step (b) occurs within a time of less than 5 minutes of step (a).

8. A method as claimed in claim 1, wherein step (b) occurs within a time of less than 10 seconds of step (a).

9. A method as claimed in claim 1, wherein step (b) is substantially simultaneous with step (a).

10. A method as claimed in claim 1, wherein further comprising adding the solution to the botanical material by at least one method selected from the group consisting of: spraying; dipping, pouring over; and combinations thereof.

11. A method as claimed in claim 1, wherein the solution is a mixture comprising at least one of: water; citric acid; ascorbic acid; cysteine; cinnamic acid; sulphur dioxide; and vitamin E.

12. A method as claimed in claim 1, wherein the solution comprises about: 0.5-30% (by weight) citric acid; 0.5-30% (by weight) ascorbic acid; and water.

13. A method as claimed in claim 1 wherein the phenolic compound is a phenolic acid.

14. A method as claimed in claim 1, wherein the solution in comprises water that has been deoxygenated.

15. A method as claimed in claim 1 wherein said liquid extract is separated from said residue within about 7 days after said mixing step.

16. A method as claimed in claim 1: further comprising separating said liquid extract from said solid residue by at least one method consisting of filtration; super critical fluid extraction (SCFE); mechanical de-watering; and a combination thereof.

17. A method as claimed in claim 16 further comprising separating said liquid extract from said solid residue by a combination of filtration and mechanical de-watering.

18. A method as claimed in claim 1 further comprising collecting a liquid extract as the retentate of reverse osmosis; collecting a liquid extract as the retentate of ultra filtration, or collecting a liquid extract as the retentate of a combination of these methods.

19. A method as claimed in claim 1 further comprising
after step (c), (d) drying the liquid extract from step (c) to obtain a solid extract.

20. A method as claimed in claim 1 further comprising, after step (c), step (e) pressing the solid residue to obtain a pressate liquid and combining said pressate with the liquid extract of step (c).

21. A method as claimed in claim 20, further comprising step (g) drying said filtrate and pressate liquid.

22. A method as claimed in claim 1 further comprising conducting at least one claims, wherein any one or a combination of steps (a) to (g) in the substantial absence of oxygen.

23. A method as claimed in claim 22, further comprising conducting at least one of steps (a) through (g) under wherein a nitrogen atmosphere.

24. A method as claimed in claim 21 further comprising at least one the further step, after step (b) and the sequences of steps selected from steps (c), (d), (e), (f) or (g), said step being:
(h) one or more further cycles of at least one of steps selected from the group consisting of steps: (b), (c), (d), (e), (f), and (g).

25. A method as claimed in claim 1, wherein the phenolic acid level after extraction is maintained at 50-100% as compared with the starting levels, measured as dried extract.

26. A method as claimed in claim 25, wherein the phenolic acid level after extraction is maintained at 70-100% of starting levels.

27. A method of extraction of biologically active compounds from Echinacea plant maternal including the steps of:
(a) reducing the particle size of freshly harvested or frozen Echinacea plant material;
(b) mixing the reduced particle size Echinacea material of step (a) with a solution comprising water, an acid compound, and an anti-oxidant compound, wherein the acid and anti-oxidant compounds are different compounds to form a liquid extract and a solid residue;
(c) separating the resulting liquid extract from the solid residue;
wherein the composition of said solution of step (b) is adapted to substantially slow or halt oxidative degradation of caftaric acid and/or cichoric compounds in the liquid extract.

28. A method of extraction of biologically active compounds from Echinacea plant material including the steps of
(a) reducing the particle size of freshly harvested or frozen harvested Echinacea plant material;
(b) mixing the Echinacea material of step (a) with a solution comprising water, citric acid, and ascorbic acid to form a liquid extract and a solid rewsidue;
(c) separating the resulting liquid extract from the solid residue;
wherein the composition of the solution of step (b) is adapted to substantially slow or halt oxidative degradation of phenolic acid compounds such that the phenolic acid level in the extract is maintained at 50-100% as compared with pre-harvest levels (measured as a dried extract) of said compounds in said plant material.

29-31. (Canceled)

32. A pressate liquid product produced by the method as of claim 20.

33. A solidified product produced by the method of claim 1.

34. A liquid extract produced by the method of claim 27.

35. A liquid extract produced by the method of claim 28.

36-43. (canceled)

44. A method as claimed in claim 13 wherein the phenolic compound is a phenolic acid

45. A method as claimed in claim 44, wherein the phenolic acid is at least one member selected from the group consisting of caftaric acid; cichoric acid.

46. A method as claimed in claim 1, wherein the solution comprises water that has been chilled to between 1 and 4° C.

47. An aqueous solution of at least one acid and at least one anti-oxidant, wherein said solution has the property of at least substantially slowing oxidative degradation of a phenolic acid compound found in plant material.

48. A liquid comprising water, an organic acid, and organic antioxidant and at least one biologically active compound extracted from plant material.

49. A product produced by drying to solid form the extract produced by the method of claim 19.

50. A method of extracting at least one biologically active compound from a botanical containing said material comprising extracting said biologically active material by contact with an aqueous solution comprising at least one acid and at least one anti-oxidant.

51. A method as a claimed in claim 50 wherein said acid is an organic compound.

52. A method as claimed in claim 50 wherein said anti-oxidant is an organic compound.
